(19) United States
(12) Patent Application Publication
(10) Pub. No.: US 2019/0060341 A1
(43) Pub. Date: Feb. 28, 2019

(54) AGAVACEAE EXTRACT COMPRISING STEROIDAL SAPONINS TO TREAT OR PREVENT METABOLIC DISORDER RELATED PATHOLOGIES

(71) Applicants: INSTITUTO TECNOLÓGICO Y DE ESTUDIOS SUPERIORES DE MONTERREY, Monterrey, Nuevo León (MX); INSTITUTO NACIONAL DE CIENCIAS MÉDICAS Y NUTRICIÓN SALVADOR ZUBIRÁN, Mexico, D.F. (MX); AGMEL S.A. DE C.V., Monterrey, Nuevo León (MX)

(72) Inventors: Ana María LEAL DÍAZ, Monterrey, Nuevo León (MX); Janet Alejandra GUTIÉRREZ URIBE, Monterrey, Nuevo León (MX); Nimbe TORRES Y TORRES, Mexico, D.F. (MX); Armando Roberto TOVAR PALACIO, Mexico, D.F. (MX); Lilia Guadalupe NORIEGA LÓPEZ, Mexico, D.F. (MX)

(73) Assignees: INSTITUTO TECNOLÓGICO Y DE ESTUDIOS SUPERIORES DE MONTERREY, Monterrey, Nuevo León (MX); INSTITUTO NACIONAL DE CIENCIAS MÉDICAS Y NUTRICIÓN SALVADOR ZUBIRÁN, Mexico, D.F. (MX); AGMEL S.A. DE C.V., Monterrey, Nuevo León (MX)

(21) Appl. No.: 15/768,674

(22) PCT Filed: Oct. 16, 2015
(86) PCT No.: PCT/IB15/02111

§ 371 Date: Apr. 16, 2018

(51) Int. Cl. A61K 31/7048; A61K 36/88; A61P 3/00; A61P 3/06; A61P 3/10; A61P 1/14; A61K 45/06; A61P 3/04

(52) U.S. Cl. CPC A61K 31/7048; A61K 36/88; A61P 3/00; A61P 3/04; A61P 3/10; A61P 1/14; A61K 45/06; A61P 3/06

(57) ABSTRACT

A saponin and sapogenin extract recovered from plants of the Agavaceae family in the form of an extract or its purified form which has beneficial effects on the organism of mammals in relation to the prevention or treatment of metabolic disorders such as obesity, metabolic syndrome, diabetes and their related pathologies in mammals, including humans and further beneficial effects on lipid metabolism, glucose metabolism, energy expenditure, and gut microbiota health. Other aspects of the invention comprise a composition made of said saponin and sapogenin extract and methods for using said extract.

### BACKGROUND OF THE INVENTION

### Field of the Invention

The present invention relates to the use of steroidal saponins and sapogenins recovered from plants of the Agavaceae family in the form of an extract or its purified form, in the preparation of a composition to be administered orally to treat or prevent metabolic disorder related pathologies in mammals.

### Problem Definition

Several metabolic disorders are a current public-health problem on the rise. These disorders or conditions are characterized by abnormal weight gain, energy use or consumption, altered metabolism of carbohydrates, lipids proteins, nucleic acids or a combination. Examples of metabolic disorders include but are not limited to metabolic syndrome, insulin resistance, insulin deficiency, type 2 diabetes mellitus, glucose intolerance, hyperglycemia, accumulation of visceral adipose tissue, adipocyte hypertrophy, hyperleptinemia, non-alcoholic fatty liver disease, hepatic steatosis, brown adipose tissue deterioration, impaired thermogenesis, dyslipidemia, mitochondria dysfunction, impaired muscle oxidative capacity, cardiovascular disease, inflammatory and immune disorders.

A main metabolic disorder is the metabolic syndrome (MetS), is a cluster of risk factors associated with cardiovascular disease (CVD), type 2 diabetes mellitus, stroke and kidney disease. It is a major worldwide clinical challenge that affects 20-40% of the world's adult population (Grundy, 2015). In United States, data from the National Health and Nutrition Examination Survey (NHANES) 2003-2012 revealed that 33% of the population above 20 years old had MetS (Aguilar, Bhuket, Torres, Liu, & Rj, 2015).

According to the National Cholesterol Education Program Adult Treatment Panel III (ATP III), MetS is defined as the presence at least three of the following five metabolic disorders: central obesity measured as the waist circumference >90 cm in man and >80 cm in woman, hyperglycemia measured as fasting glucose >100 mg/dL, hypertriglyceridemia measured as blood triglycerides <150 mg/dL, low plasma high density lipoprotein cholesterol (HDL-C)<40 mg/dL in man and <50 mg/dL in woman and hypertension measured as the blood pressure ≥130/85.

People with MetS have a 5-fold greater probability to develop type 2 diabetes, and 80% of the world's diabetic population (200 million) will die from cardiovascular diseases. According to the international diabetes foundation, diabetes prevalence in 2014 was 11.4% in United States, and the estimated cost per person was USD $10,900.

The increasing diabetes prevalence is influenced by the also rapidly growing obesity, which is considered the most important risk factor for type 2 diabetes. About 90-95% of type 2 diabetes is attributable to excess weight (Geiss et al., 2014). Obesity is categorized using the Body Mass Index (BMI) equation. It is obtained by dividing the body mass or weight by the square of the individuals height (kg/m2). A healthy weight individual has a BMI between from 18.5 and 25 kg/m², overweight from 25 to 30 kg/m², obese class I from 30 to 35 kg/m², obese class II from 30 to 35 kg/m² and obese class III over 40 kg/m². Obesity itself increases the likelihood to develop metabolic syndrome, hypertension, type 2 diabetes, non-alcoholic fatty liver disease, or obesity-related kidney disease (Grundy, 2015).

White adipose tissue (WAT) is the tissue where excess nutrients are stored in the form of lipids in unilocular adipocytes when overfeed, so they may be released as fatty acids to be utilized as energy when food scarce (Bartelt & Heeren, 2014). Surplus fat is stored either by increasing the size of the adipocytes (hypertrophy) or number of adipocytes (hyperplasia) (Grundy, 2015). Fat may be stored in the different parts of the body, the lower body fat is stored subcutaneously in the legs and hips and the upper body fat may also be stored subcutaneously but additionally inside the abdominal cavity between the organs. Upper body fat is also referred as visceral fat and it is more related with MetS and hepatic steatosis compared with lower body fat.

The adipose tissue is now recognized as an endocrine tissue capable to secrete hormones or adipokines influencing systemic metabolism and appetite. Dysfunctional enlarged adipose tissue with hypertrophic adipocytes produces more pro-inflammatory factors and less anti-inflammatory factors. Obesity also impairs leptin secretion by the WAT (Pan, Guo, & Su, 2014). Leptin regulates energy metabolism by increasing energy expenditure and decreasing energy intake and it is considered a metabolic signal for energy sufficiency. Unfortunately, during obesity, leptin resistance is developed which may evolve to hyperleptinemia (Pan et al., 2014). Some phytochemicals, such as isoflavones, may be used to prevent or treat hyperleptinemia (WO2012/145281).

Diabetes is a metabolic disease characterized by hyperglycemia caused by defect on insulin secretion, insulin action or both (American Diabetes Association., 2014).

Insulin resistance precedes diabetes and it is a physiological condition when even though insulin can be normally secreted, the cell responds inefficiently to the normal insulin stimulation impairing glucose uptake and causing hyperglycemia. The pancreas reacts to this condition by secreting more insulin in order to prevent hyperglycemia. The high concentration of insulin is referred as hyperinsulinemia.

Skeletal muscle is the primary tissue to use the glucose in the postprandial state and during exercise. Patients with insulin resistance show skeletal muscle with less type 1 oxidative fibers compared to the type 2 glycolytic fibers (Lagouge et al., 2006). Skeletal muscle biopsies from type 2 diabetes patients have also lower oxidative phosphorylation capacity compared with healthy individuals and a decreased type 2 fibers which are rich in mitochondria. Mitochondrial dysfunction has been observed in animals with lower aerobic capacity and decreased expression of genes related to mitochondrial biogenesis and oxidative phosphorylation, especially the transcriptional co-activator called peroxisome proliferator-activated receptor gamma coactivator-1alpha (PGC- [symbol] ) (Lagouge et al., 2006). PGC- [symbol] affects many biological pathways related to energy metabolism. In the muscle, it regulates mitochondrial biogenesis and triggers angiogenesis as well as production of oxidative fibers. PGC- [symbol] is activated by endurance exercise or the activation of AMP-activated kinase (AMPK). The activation of AMPK in the muscle increases glucose uptake by increasing the glucose transporter 4 (GLUT4) translocation, fatty acid oxidation, and mitochondrial function and biogenesis (Jager, Handschin, St. Pierre, & Spiegelman, 2007). Triterpenic saponins have been patented to treat metabolic syndrome or decrease obesity due to their effect on the regulation of AMPK activity (U.S. Pat. No. 8,357,786B2, US 2014/0141108A1).

PGC- [symbol] acts in other tissues such as adipose tissue where it activates the mitochondrial uncoupling protein 1 (UCP1) and thermogenesis through irisin (Boström et al., 2012). Resveratrol and certain flavonoids stimulate mitochondrial function by PGC- [symbol] regulation and new compounds have been synthesized to enhance muscle oxidative capacity by this route (RU2559779-C1).

In mammals the main thermogenic tissue is the brown adipose tissue (BAT) that dissipate energy as heat in a process called non-shivering thermogenesis (Bartelt & Heeren, 2014). Its activation confers beneficial effects on adiposity, insulin sensitivity and hyperlipidemia (Bartelt & Heeren, 2014). Different from white adipose tissue, BAT is characterized by a multilocular lipid droplet structure with high amounts of mitochondria and UCP-1. Browning is referred as the process where WAT adipocytes, especially subcutaneous, express UCP1 and it is mainly activated by cold exposure and endurance exercise through PGC. [symbol] and irisin (Harms & Seale, 2013). On the other side, enlarged BAT adipocytes with a unilocular lipid droplet may indicate impaired thermogenesis and lipid oxidation.

Metabolic syndrome and diabetes are commonly associated with non-alcoholic fatty liver disease (NAFLD). The first NAFLD stage is hepatic steatosis; an accumulation of intracytoplasmatic triacylglycerides (TAG) in the hepatocyte. Alanine aminotransferase (ALT) enzyme is elevated in the blood during hepatic steatosis or NAFLD and consequently it has been used as a marker for this disease (Schindhelm et al., 2009). This enzyme is also used in routine preclinical safety assessment studies as a biomarker of hepatotoxicity.

Intestinal or gut microbiota is the collective microbial community inhabiting this environment (Tremaroli & Backhed, 2012). Gut microbiota modification or dysbiosis is nowadays widely recognized to be linked to different metabolic disorder related pathologies such as MetS and its associated disorders including NAFLD. Some of the gut microbiota alteration to the host include: energy balance alteration, gut permeability, metabolic endotoxemia, and inflammation, which are all associated to obesity and its disorders (Everard et al., 2013). Changes in microbiota are also related to metabolic health improve.

Intestinal microbiota is conformed of there main phyla; Bacteroidetes, Firmicutes and Proteobacteria (Ley, Turnbaugh, Klein, & Gordon, 2006). The phyla proportion is altered with the dietary habits. Particularly, by the consumption of prebiotics, which are defined as non-digestible food constituents that selectively stimulate the activity or growth of specific bacteria producing a benefit to the host (Roberfroid, 2000). It has been recently demonstrated that saponins found in herbal preparations have beneficial effects on the gut microbiota (Chen, Tai, & Hsiao, 2015), nevertheless in that study the metabolic parameters were not evaluated to assess if the change in microbiota exerted a benefit to the host.

During obesity, Firmicutes is predominant over Bacteroidetes, and upon diet intervention the opposite occurs (Ley et al., 2006). Probiotic bacteria have been evaluated for their ability to modulate obesity. Bifidobacterium has shown to decrease endotoxin levels and improved intestinal mucosal barrier function (Cani et al., 2007). The oral consumption one strain of Lactobacillus reuteri on DIO model proved that it prevented weight gain, decreased hepatic steatosis, and increased CPT1a expression in the liver suggesting increased 0-oxidation (Fåk & Bäckhed, 2012). Nevertheless the authors did not reported if the Lactobacillus consumption modified its abundance in the intestinal microbiota. The U.S. Pat. No. 8,440,178B2 claims that Lactobacillus rhamnosus CGMCC 1.3724 and/or Lactobacillus rhamnosus NCC 4007 promote weight loss and/or to treat obesity. A different bacteria, Akkermansia muciniphila, is a mucin-degrading bacteria that resides in the intestinal mucus layer and which has been associated with an improvement of insulin sensitivity and a decrease in fat gain in obese mice (Everard et al., 2013). In obese humans, higher A. muciniphila abundance prior a calorie restriction diet was associated with a greater glucose tolerance increase and decrease in LDL and total cholesterol (Dao et al., 2015). The patent No. WO2014076248A1 claims the use of A. muciniphila to treat metabolic disorders, promote weight loss and increase energy expenditure when it is orally consumed.

The previously described pathologies belong to metabolic disorder related pathologies.

### Description of the Related Art

Different drugs to decrease weight are available in the market. The majority of the new drugs to decrease the weight target an appetite decrease or satiety increase. Orlistat from Xenical is the exception, it decreases fat absorption by inhibiting the pancreatic lipases, but with the side effect of decreased fat-soluble vitamins absorption and steatorrhea (Apovian et al., 2015; Yun, 2010). Lorcaserin commercially known as Belviq (U.S. Pat. No. 7,514,422B2) increases satiety through increasing the serotonin 2C receptor but with side effects of headache, nausea and constipation (Apovian et al., 2015). A combination of phentermine and topiramate, commercially known as Qsyrnia, stimulates the central nervous system similar amphetamines but has serious side effects such has birth defects (Apovian et al., 2015). Contrave combines bupropion and naltrexone, increasing satiety and energy expenditure, but has the side effect of possible suicidal thoughts or actions. Finally, Victoza contains liraglutide is a glucagon-like protein 1 (GLP1-) receptor agonist and bedsides increasing insulin secretion, it suppresses appetite and decreases food intake (U.S. Pat. No. 6,458,924B2) nevertheless, the side effects include nausea, vomiting, diarrhea and pancreatitis (Apovian et al., 2015).

Because of discontent of harmful side-effects, the potential of natural products to prevent or decrease obesity is under examination. Natural products to treat obesity have four distinct mechanisms: (1) decreased lipid absorption by inhibiting the pancreatic lipases, (2) decreased energy intake, (3) increased energy expenditure, (4) decreased pre-adipocyte differentiation and proliferation (Yun, 2010). Polyphenols, flavonolds, phytoesterols and saponins are associated to decrease or treat obesity (Santos, Rogero, & Bastos, 2010; Yun, 2010). Many research articles and patents disclose the effect of saponins to prevent or treat metabolic disorder such as MetS, type 2 diabetes and its related pathologies. These molecules may be isolated, in a crude extract or as part of a composition.

Saponins from Panax ginseng, Panax japonicas, and Platycodi radix been validated in different models to prevent or decrease obesity. Saponins from Panax ginseng have been reported to suppress appetite therefore decreasing weight gain. For example a crude saponin extract resulted in a 37% decreased weight gain in mice (Yun, 2010). A further study in rats showed that the saponin Ginsenoside Rb1 from this same plant was an active saponin (Xiong et al., 2010).

Saponins from Panax ginseng have also reported an increased energy expenditure causing a decrease in body weight. For example an ethanolic crude extract from the Panax ginseng berry increased energy expenditure and decreased body weight by 13% (Attele et al., 2002). The Ginsenoside Rb1 also increased the energy expenditure and promoted weight reduction in mice (Xiong et al., 2010).

Muscular health is also involved in the energy metabolism improvement. Ginseng saponins have also been used to improve the muscular strength and energy such as in the U.S. Pat. No. 6,485,018B1. Also, a methanolic extract of the root of Platycodon grandiflorum ameliorated obesity and insulin resistance by activating the AMPK/ACC phosphorylation in vitro in C2C12 myotubes and decrease lipid accumulation in 3T3-L1 adipocytes (Lee et al., 2012).

The U.S. Pat. No. 7,985,848B2 claims a pharmaceutical composition for preventing and treating diabetes or glucose control abnormality comprising the triterpenic saponins gingenosides Rg3, Rg5, and Rk1. The Chinese Pat. No. CN102,091,082B relates to gingenoside Rb3 triterpenic saponin in preparing a medicament for treating diabetes.

Most of the studied saponins belong to the triterpenic structure consisting on 30 carbons in the aglycone such as the saponins extracted from Panax ginseng, Panax japonicas, Platycodon grandiflorum previously described. The present invention is different because it involves the use of steroidal saponins that have a 27 carbon aglycone.

Dioscin is a steroidal saponin that has also been proven to decrease obesity and increase the energy expenditure (Liu et al., 2015). This saponin may be extracted from different sources such as Dioscorrea app. and Trigonella foenum-graecum. Its aglycone is diosgenin and diosgenin glycosides have been report in Agave britoniana (Macias, Guerra, Simonet, & Nogueiras, 2007). However the researchers did not evaluate other biological effect of this compound in mammalians, moreover, in the present invention Dioscin was not present.

In Dioscorea polygonoides, diosgenin has been proposed to be the active component responsible for the beneficial effects to treat hyperglycemia (Omoruyi, 2008). Trigonella foenumgraecum seed powder, also rich in diosgenin, has also been evaluated in clinical trials. Its consumption was evaluated in type 2 diabetes patients where results showed blood sugar reduction (Mitra & Bhattacharya, 2006). A furostanolic-saponin rich fraction extracted from Trigonella foenumgraecum seeds with >30% of protodioscin decreased blood glucose when consumed daily (WO2010/140165A1). Also a composition with spirostan steroidal saponins extracted from Chlorophytum arundinacceum from the Liliaceae family was claimed to decrease weight and dyslipidemia (US2006/0062863A1) (USDA, 2015).

The previous patents and research articles have showed the state of the art in the use of saponins to treat different diseases related to a metabolic disorder including metabolic syndrome and type 2 diabetes. However, there are no previous reports on the use of saponins from the Agavaceae family for such effect. More specific there is no information regarding its effect to prevent or decrease obesity, hyperglycemia, insulin resistance, visceral adipose tissue accumulation, hepatic steatosis, dyslipidemia, adipocyte size or to improve microbiota health, increase oxidative muscle fibers, mitochondria abundance and/or thermogenesis.

The Agavaceae family holds 10 genera which are Agave L., Yucca L., Dracaena L., Furcraea Vent., Hesperaloe Engelm., Hesperoyucca (Engelm.) Baker, Manfreda Salisb, Phormium J. R. Forst & G. Forst, Polianthes L., and Sansevleria Thunb (USDA, 2015).

Particularly the genus Agave holds more than 300 species many of which have their center of origin in Mexico and grow natively in arid and tropical regions. Anatomically, these plants are rosettes with two main aerial parts: the long spiked leaves and the stem or “piña” from which sweet agave sap called “aguamiel” can be collected (Leal-Díaz et al., 2015). Agave is a succulent plant, therefore it loses minimum water during drought, and uptakes water rapidly when drought ceases, for this reason it can store a vast amount of sap.

Since pre-Columbian times agave has been used as a source of food and beverage, mainly derived from its sap, the stem called “quiote” or the flowers (Santos-Zea, Leal-Díaz, Cort\tilde{e}s-Ceballos, & Gutiérrez-Uribe, 2012). The sap itself when extracted from mature agaves is consumed fresh as the beverage “aguamiel”. Mexican pre-Hispanic cultures also used agave for medicinal purposes but very little is known about the active ingredients.

Currently the most studied components from agave are the agave fructans or inulin. The U.S. Pat. No. 7,812,004B2 claims the use of inulin that may be obtained from agave with the objective to increase the bone mineralization and to prevent or treat osteoporosis.

Different studies using agave fructans have shown an effect in body weight. The Pat. No. WO2012/066485A2 claims a decrease in body weight with a composition made of agave fructans. When the fructans were added at 15% of the diet, hepatic steatosis and LDL-cholesterol were also decreased (Rendón-Huerta, Juárez-Flores, & Delgado-Portales, 2012). In this same study the beneficial intestinal bacteria Bifidobacterium spp. and Lactobacillus spp. where increased. Some of these results concur with the present invention; nevertheless different from the reports on agave fructans, the extract in the present invention contains steroidal saponins.

Steroidal saponins have been identified in agave leaves, rhizomes and “aguamiel” (Leal-Díaz et al., 2015; Santos-Zea at al., 2012). In aguamiel, eight different saponins were reported mainly derived from the sapogenins kammogenin, manogenin, gentrogenin and hecogenin.

Saponins and sapogenins have been recently studied mainly for their cytotoxic properties. The U.S. Pat. No. 8,470,858B2 claims an extract with sapogenins obtain from agave syrup that inhibits cancer cells growth and with some antioxidant properties.

However, until now, there have not been any attempts to use steroidal saponins extracted from the Agavaceae family for the prevention or treatment of metabolic disorders including metabolic syndrome, diabetes and their related pathologies.

Applicants discovered that an Agavaceae extract containing steroidal saponins, as those found in Agavaceae plants, have potential effects to prevent or treat metabolic disorder pathologies such as metabolic syndrome and type 2 diabetes, more specifically to decrease hyperglycemia, insulin resistance, visceral adipose tissue accumulation, hepatic steatosis, dyslipidemia, body weight gain and adipocyte size or to improve gut microbiota health, promote Akkermansia muciniphila gut abundance, and increase muscle oxidative fibers, mitochondrial activity and thermogenesis.

In view of the above, applicant obtained steroidal saponins recovered from plants of the Agavaceae family in the form of an extract or its purified form, for the preparation of a composition to be administered orally to treat or prevent metabolic disorder pathologies in mammals.

The composition is useful to prevent or treat overweight and obesity, hyperglycemia, insulin resistance, visceral tissue accumulation, hepatic steatosis, dyslipidemia and adipocyte hypertrophy. Furthermore, the composition is useful to improve the intestinal microbiota health, promote Akkermansia muciniphila intestinal abundance, and increase muscle oxidative capacity, energy expenditure, mitochondrial activity and thermogenesis.

The extract can be profitably used in the food and beverage industry as an ingredient to formulate a functional food or beverage, or food or beverage supplement. It can also be advantageously used in the pharmaceutical industry as part of a pharmaceutical composition to prevent or treat the pathologies early mentioned.

### SUMMARY OF THE INVENTION

It is therefore a main object of the present invention to provide steroidal saponins and sapogenins recovered from plants of the Agavaceae family in the form of an extract or its purified form which has beneficial effects on the organism of mammals in relation to the prevention or treatment of metabolic disorder related pathologies.

It is another main object of the present invention; to provide a composition comprised by steroidal saponins and sapogenins recovered from plants of the Agavaceae family in the form of an extract or its purified form to be administered orally to treat or prevent metabolic disorder related pathologies in mammals.

It is another object of the present invention to provide a method to treat or prevent metabolic disorder related pathologies in mammals comprising administering to mammals steroidal saponins and sapogenins recovered from plants of the Agavaceae family in the form of an extract or its purified form.

It is a further main object of the present invention, to provide a composition comprised by steroidal saponins and sapogenins recovered from plants of the Agavaceae family in the form of an extract or its purified form of the above referred nature, which is useful to prevent or treat overweight and obesity, hyperglycemia, insulin resistance, visceral adipose tissue accumulation, hepatic steatosis, dyslipidemia and adipocyte hypertrophy, as well to improve the intestinal microbiota health, increase Akkermansia muciniphila intestinal abundance, muscle oxidative capacity, energy expenditure, mitochondrial activity and thermogenesis.

It is another main object of the present invention to provide steroidal saponins and sapogenins recovered from plants of the Agavaceae family in the form of an extract or its purified form of the above referred nature, which can also be advantageously used in the pharmaceutical industry as part of a pharmaceutical composition to prevent or treat the pathologies early mentioned.

It is another main object of the present invention to provide steroidal saponins recovered from plants of the Agavaceae family in the form of an extract or its purified form of the above referred nature, which can also be advantageously used in the food and beverage industry as part of a functional beverage or food composition or a food or beverage supplement to prevent the pathologies early mentioned.

These and other objects and advantages of the present invention will become apparent to those persons having an ordinary skill in the art, from the following detailed description of the invention, which will be made with reference to the accompanying figures.

### BRIEF DESCRIPTION OF THE FIGURES

FIG. 1. Shows the chemical structure of the sapogenin kammogenin, manogenin, gentrogenin and hecogenin

FIG. 2. Shows a HPLC-ELSD chromatogram depicting a steroidal saponin enrichment strategy. (1) Kammogenin glycosides, (2) manogenin glycosides, (3) gentrogenin glycosides and (4) hecogenin glycosides.

FIG. 3. Shows a graph contrasting the weight change in mice fed control diet (C) and high fat diet (HF) supplemented with agave sap concentrate (HFSC), or low dose of saponin extract (HFLS) or high dose of saponin extract (HFHS) during 80 days.

FIG. 4. Shows the mice weight gain (g) after 16 weeks of feeding a high fat diet (HF) in contrast to those fed control diet (C) or high fat diet supplemented with agave sap concentrate (HFSC), or low dose of saponin extract (HFLS) or high dose of saponin extract (HFHS). Weight gain of mice fed HFHS was even lower than the observed in mice fed control diet.

FIG. 5. Shows the effect on the body weight (g) of mice fed a HF diet during the first 16 weeks and afterwards a HF diet supplemented with a saponin extract (HFHS). The diet switch decreased body weight by 28% after 8 weeks of feeding HFHS.

FIG. 6. Blood glucose concentration (mg/dL) during an oral glucose tolerance test of mice fed the HF diet during 16 weeks (OGTT1) and after the switch to HFHS diet during 8 weeks (OGTT2). * P<0.05, ** P<0.005.

FIG. 7. Area under the curve obtained from the oral glucose tolerance test of mice fed the HF diet during 16 weeks (OGTT1) and after the switch to HFHS diet during 8 weeks (OGTT2).

FIG. 8. Shows the different adipocyte size of mice white adipose tissue histological cuts stained with hematoxylin & eosin of (A) epididymal visceral adipose tissue and (B) subcutaneous adipose tissue after 12 weeks of feeding control diet (C) or high fat diet (HF) supplemented with agave sap concentrate (HFSC), or low dose of saponin extract (HFLS) or high dose of saponin extract (HFHS).

FIG. 9. Shows mice liver histological cuts stained with hematoxylin & eosin (A) and Oil Red O (B) after 12 weeks of feeding control diet (C) or high fat diet (HF) supplemented with agave sap concentrate (HFSC), or low dose of saponin extract (HFLS) or high dose of saponin extract (HFHS).

FIG. 10. Shows the contrast of hepatic triacylglycerides or triglycerides (TAG) concentration (mg/g) in mice fed during 12 weeks a high fat diet (HF) against control diet (C) or high fat diet supplemented with agave sap concentrate (HFSC) or low dose of saponin extract (HFLS) or high dose of saponin extract (HFHS). ** P<0.005, ***P<0.001.

FIG. 11. Shows the contrast of blood plasma alanine aminotransferase (ALT) concentration (U/L) in mice fed during 12 weeks a high fat diet (HF) against control diet (C) or high fat diet supplemented with agave sap concentrate (HFSC) or low dose of saponin extract (HFLS) or high dose of saponin extract (HFHS). ** P<0.005, ***P<0.001.

FIG. 12. Shows the contrast of blood plasma leptin concentration (ng/mL) in mice fed during 12 weeks a high fat diet (HF) against control diet (C) or high fat diet supplemented with agave sap concentrate (HFSC) or low dose of saponin extract (HFLS) or high dose of saponin extract (HFHS). ***P<0.001.

FIG. 13. Shows blood glucose concentration (mg/dL) during an oral glucose tolerance test of mice fed control diet (C) or high fat diet (HF) or high fat diet supplemented with agave sap concentrate (HFSC), or low dose of saponin extract (HFLS) or high dose of saponin extract (HFHS) during 10 weeks.

FIG. 14. Shows the contrast of blood plasma LDL-cholesterol concentration (mg/dL) in mice fed during 12 weeks a high fat diet (HF) against control diet (C) or high fat diet supplemented with agave sap concentrate (HFSC) or low dose of saponin extract (HFLS) or high dose of saponin extract (HFHS). *P<0.05, ** P<0.005, ***P<0.001.

FIG. 15. Shows mRNA relative expression of the low density lipoprotein receptor (LDLr) in the liver of mice fed control diet (C) or high fat diet (HF) or high fat diet supplemented with agave sap concentrate (HFSC), or low dose of saponin extract (HFLS) or high dose of saponin extract (HFHS) during 12 weeks. Columns with different letters are significantly different (P<0.05).

FIG. 16. Shows oxygen consumption (mL/Kg/h) at fasting and feeding stage of mice fed control diet (C) or high fat diet (HF) or high fat diet supplemented with agave sap concentrate (HFSC), or low dose of saponin extract (HFLS) or high dose of saponin extract (HFHS) during 11 weeks. Columns with different letters are significantly different (P<0.05)

FIG. 17. Shows (A) soleous skeletal muscle and (B) gastrocnemius skeletal muscle succinate dehydrogenase (SDH) histochemistry staining to distinguish the oxidative muscle fibers in mice fed a high fat diet (HF) against control diet (C) or high fat diet supplemented with agave sap concentrate (HFSC) or low dose of saponin extract (HFLS) or high dose of saponin extract (HFHS) during 12 weeks. Black arrows indicate highly oxidative muscle fibers.

FIG. 18. Shows the thermogenic brown adipose tissue histological cuts stained with hematoxylin and eosin of mice fed a high fat diet (HF) against control diet (C) or high tat diet supplemented with agave sap concentrate (HFSC) or low dose of saponin extract (HFLS) or high dose of saponin extract (HFHS) during 12 weeks.

FIG. 19. Shows white adipose tissue browning effect observed by the expression of mitochondrial uncoupling protein 1 (UCP-1) detected by immunohistochemistry staining in the (A) epididymal visceral adipose tissue and (B) subcutaneous adipose tissue of mice fed a high fat diet (HF) against control diet (C) or high fat diet supplemented with agave sap concentrate (HFSC) or low dose of saponin extract (HFLS) or high dose of saponin extract (HFHS) during 12 weeks.

FIG. 20. Shows the overexpression of the peroxisome proliferator-activated receptor gamma coactivator 1-0 (PGC-10) in skeletal muscle protein of mice fed a high fat diet supplemented with different dose of saponins extract (HFLS or HFHS) during 12 weeks. *** P<0.001.

FIG. 21. Shows the increase in skeletal muscle phosphorylation ratio of the enzyme 5′ adenosine monophosphate-activated protein kinase (P-AMPK/AMPK) in mice fed a high fat diet supplemented with saponins extract (HFHS) during 12 weeks. * P<0.05.

FIG. 22. Shows the increase in relative abundance of the beneficial fecal bacteria Bifidobacterium spp. after feeding mice with a high fat diet with different concentrations of saponins (HFLS or HFHS) during 12 weeks. Columns with different letters are significantly different (P<0.05)

FIG. 23. Shows the increase in relative abundance of the beneficial fecal bacteria Akkermansia muciniphila after feeding mice with a high fat diet with different concentrations of saponins (HFLS or HFHS) during 12 weeks. Columns with different letters are significantly different (P<0.05).

### DETAILED DESCRIPTION OF THE INVENTION

The present invention will now be described in accordance with a preferred embodiment thereof and the description and examples refer to the methods and steps for using the present invention.

In one aspect, the present invention comprises a saponin and sapogenin extract from plants of the Agavaceae family in the form of a crude extract or its purified form comprising steroidal saponins at a concentration of from 30 to 90% in weight, wherein said extract has beneficial effects on the organism of mammals in relation to the prevention or treatment of metabolic disorder related pathologies in mammals.

The most abundant saponins present in the extract are kammogenin glycosides, comprising >30% of the total saponins and sapogenins combined. The sapogenin concentration present in the extract is at least 0.01%.

The extract may also contain saponins having at least one of the following aglycones or genins: kammogenin, manogenin, gentrogenin, hecogenin, tigogenin, sarsapogenin, chlorogenin and gitogenin or their corresponding isomer or oxidized or reduced forms with at least one of the following glycosidic moieties (in the form of acid or salt): glucose, xylose, rhamnose, arabinose, or galactose

The extract may contain one of the following saponins: agamenoside, agaveside, agavoside, magueyside, agavasaponi, cantalasaponin, sisalsaponin, gabrittonoside, dongnoside or amolonin, or other steroidal saponins.

The extract may contain other phytochemicals such as alkaloids, polyphenols, flavonoids, phytosterols, triterpenes, or policosanols.

The extract may be obtained from the complete plant, sap, shoots, bark, leaf, stem, root, rhizomes, flower, fruit, flower stem or callus from plants of at least one of the following species from the agave genus Yucca L.: Yucca schotti, Yucca schidigera, Yucca gloriosa L., Yucca schidigera Roezl, Yucca alofoia, Yucca whipplei, Yucca brevifolia, Yucca elate, Yucca elephantipes, Yucca filamentosa, Yucca baccata, Yucca valida.

Preferably, the extract is obtained from the complete plant or from different parts of the plant such as sap, agave leaf, stem, root, flower, fruit, flower stem or callus (preferably from the sap) from different agave species including but not limited to Agave salmiana, Agave tequilana Agave americana L., Agave angustifolia Haw., Agave atrovirens, Agave deserti Engelm, Agave utahensis Engelm, Agave offoyana, Agave ferox, Agave mapisaga, Agave durangensis, Agave amaniensis and Agave franzosini Nissen from the genus Agave L.

The sap that could be used for obtaining the extract may be fresh, boiled, concentrated, dried, distilled, fermented, lyophilized or aged.

Specifically, the extract of the present invention may be successfully used to prevent the following pathologies in mammals: obesity, overweight, accumulation of visceral adipose fat and central obesity, adipocyte enlargement or hypertrophy, fatty liver, hepatic steatosis, intracytoplasmatic hepatic triacylgliceride accumulation, non alcoholic fatty liver disease, blood transaminases increase, ALT increase, hyperteptinemia, brown adipose tissue deterioration by unilocular structure with large lipid vacuole, fasting blood glucose above 90 mg/dL, fasting blood insulin above 15 [symbol] U/mL, postprandial blood glucose above 140 mg/dL, blood glycated hemoglobin above 5%, insulin resistance characterized by HOMA >2.5, dyslipidemia, blood triacylglycerides concentration above 150 mg/dL, blood total cholesterol concentration of 240 mg/dL, prevent blood LDL-cholesterol concentration above 100 mg/dL, blood HDL-cholesterol concentration below 40 mg/dL for males and 50 mg/dL for females, skeletal muscle type 1 fiber loss, mitochondrial dysfunction, intestinal microbiota dysbiosis by decreasing the abundance of the phyla Firmicutes and increasing the abundance of the phyla Bacteroidetes.

Furthermore, the extract of the present invention may be successfully used to provide the following benefits to mammals:

### Benefits on Lipid Metabolism:

1. Promotes total body weight loss
2. Decreases obesity
3. Decreases overweight
4. Decreases the accumulation of visceral adipose fat and central obesity
5. Decreases adipocyte enlargement or hypertrophy
6. Decreases hyperleptinemia
7. Decreases fatty liver
8. Decreases hepatic steatosis
9. Decreases intracitoplasmatic hepatic triacylgliceride accumulation
10. Decreases non alcoholic fatty liver disease
11. Decreases ALT concentration
12. Decreases blood transaminase concentration
13. Decreases brown adipose tissue deterioration by unilocular structure with large lipid vacuole
14. Decreases dyslipidemia
15. Decreases blood triglycerides
16. Decreases Total cholesterol
17. Decreases LDL cholesterol
18. Decreases HDL cholesterol
19. Decreases Mitochondrial dysfunction

### Benefits on Glucose Metabolism

1. Decreases fasting glucose
2. Decreases fasting blood insulin
3. Decreases postprandial blood glucose
4. Decreases glycated hemoglobin
5. Decreases Insulin resistance

### Benefits on Energy Expenditure

1. Increases skeletal muscle type 1 oxidative fibers
2. Increases mitochondrial abundance in the muscle
3. Increases PGC- [symbol] , UCP1, and AMPK activation
4. Decreases mitochondrial dysfunction
5. Increases thermogenesis
6. Increases oxygen consumption

### Benefits on Gut Microbiota Health

1. Prevents microbiota dysbiosis
2. Increases Bacteroidetes
3. Decreases Firmicutes
4. Increases Akkermansia muciniphila abundance
5. Increases Lactobacillus spp. abundance
6. Increases Bifidobacterium spp. abundance

With regard to weight loss, which is one of the beneficial effects of the extract of the present invention, the fat excretion in the feces was not increased when administering the extract to a mammal, suggesting that the pancreatic lipases are not inhibited, furthermore, the energy expenditure was increased causing a decrease in body weight and improvements in different metabolic parameters

The extract of the present invention can be profitably used in the food industry as an ingredient to formulate a functional food or food supplement. It can also be advantageously used in the pharmaceutical industry as part of a pharmaceutical composition to prevent or treat the pathologies early mentioned.

In other aspect, the present invention, comprises a composition comprised by steroidal saponins recovered from plants of the Agavaceae family in the form of an extract or its purified form as described above, to be administered orally or in any other suitable form to mammals, to treat or prevent metabolic disorder, diabetes and their related pathologies in mammals as described above, wherein the amount of extract comprises from 0.001 to 70% in weight. Preferably the amount of saponins in the composition is from 0.001 to 70% in weight.

The composition of the present invention may be successfully used to prevent the same pathologies as the ones listed above when using the extract alone and provides the same benefits as the ones listed above when using the extract alone.

The administration of the composition of the present invention in individuals with BMI from 18.5-45 Kg/m², promotes abundance of Bifidobacterium spp., Lactobacillus spp. or Akkermansia muciniphila in the intestine, increases muscle oxidative capacity mainly by increasing the type I oxidative muscle fibers, increases energy expenditure, fatty acid oxidation, oxygen consumption, mitochondrial activity and biogenesis, and thermogenesis by stimulating PGC- [symbol] and UCP1, and activating AMPK.

The composition may be administered as tablet, capsule, dragee, food or beverage, food or beverage supplement, candy, beverage, herbal remedy, homeopathy or injectable solution, powder, liquid, chewy, however, other suitable methods of administration may be used or suggested by experts in the field.

Consequently, the administration route may be oral, sublingual, buccal, injected or iv.

### EXAMPLES

The following Examples serve to further illustrate the present invention and are not to be construed as limiting its scope in any way.

### Example 1

### Steroidal Saponin Extract

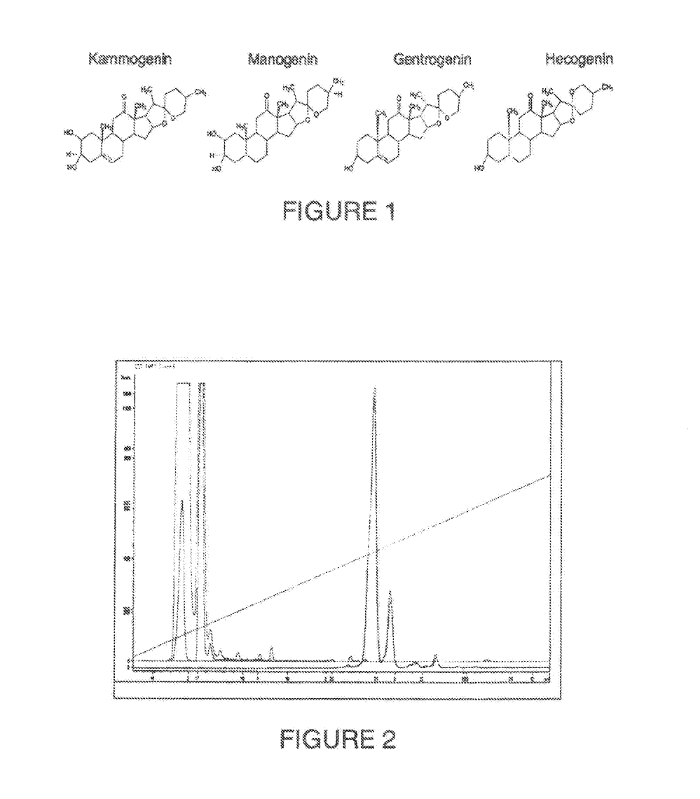

A saponin extract was obtained from agave sap concentrate using a hydrophilic polar solvent and water to increase 5 to 20 times the concentration of total saponins. Saponins were separated by HPLC and detected using an Evaporative Light Scattering Detector and analyzed by mass spectrometry. Saponins were composed of glycosides of kammogenin, manogenin, gentrogenin and hecogenin (FIG. 1). The extract contained 65.9 mg/g of saponins, from which 74% were kammogenin derivatives, 11% were derived from manogenin, 8% from gentrogenin and 7% of hecogenin. Saponin composition changed depending on the natural source in some cases reducing the abundance of kammogenin glycosides to less than 50%. When the concentration of saponins in the raw material decreased 10 times, as in an agave sap concentrate with 0.23 mg/g instead of 2.45 mg/g, the concentration in the raw extract was 40 times lower. Therefore an enrichment of the raw material is recommended before extraction. Additionally further cleaning or enrichment process may be used to obtain saponin concentrations higher than 1.6 mg/g in the extracts.

### Example 2

### Steroidal Saponin Extract Enrichment

The raw saponin extract may be enriched after serial water partitioning prior solvent evaporation. Sequential water partitioning reduced the abundance of compounds that eluted from the reverse phase column before the saponins derived from kammogenin (FIG. 2). Partitioning chromatography or ion-exchange chromatography may be used to separate or isolate saponins from other polar contaminants, mainly sugars, phenolics and amino acid derivatives.

### Example 3

A Saponin Extract that Prevents Body Weight Gain Despite the High Fat Diet Consumption

Thirty-five C57BL6 mice (5 weeks old) were assigned to five treatments (n=7) and fed ad libitum for 12 weeks. The diets are presented in Table 1.

TABLE 1
Experimental diet composition

| | C | HF | HFSC | HFLS | HFHS |
|---|---|---|---|---|---|
| Ingredient (g/Kg) | | | | | |
| Corn starch | 397.5 | 247.7 | 247.7 | 247.7 | 247.7 |
| Casein (≥85% protein) | 200 | 245 | 241.8 | 245 | 245 |
| Maltodextrin | 132 | 71.3 | 71.3 | 71.3 | 71.3 |
| Sucrose | 100 | 100 | 76.9 | 100 | 100 |
| Soybean oil | 70 | 73.5 | 73.5 | 73.5 | 73.5 |
| Cellulose | 50 | 50 | 50 | 50 | 50 |
| Minerals | 35 | 35 | 35 | 35 | 35 |
| Vitamins | 10 | 10 | 10 | 10 | 10 |
| L-Cystine | 3 | 3 | 3 | 3 | 3 |
| Choline | 2.5 | 2.5 | 2.5 | 2.5 | 2.5 |
| Lard | — | 161.5 | 161.5 | 161.5 | 161.5 |
| Saponin rich extract | — | — | — | 2.8 | 28 |
| Agave sap concentrate | — | — | 50 | — | — |
| % Energy (Kcal) from: | | | | | |
| Protein | 19 | 19 | 19 | 19 | 19 |
| Fat | 16 | 45 | 45 | 45 | 45 |
| Carbohydrates | 65 | 36 | 36 | 36 | 36 |

Control (C) diet was based on AIN-93 diet for rodents(Reeves, Nielsen, & Fahey, 1993); high-fat (HF) diet was based also on AIN-93 with 45% of the Kcal from fat; HF diet with 5% of agave sap concentrate (HFSC); HF diet with low saponin extract dose, adding 2.8 g saponin extract (dry matter)/kg diet (HFLS); HF diet with high saponin extract dose (HFHS) adding 28 g saponin extract (dry matter)/kg diet. Animals were weighted twice a week.

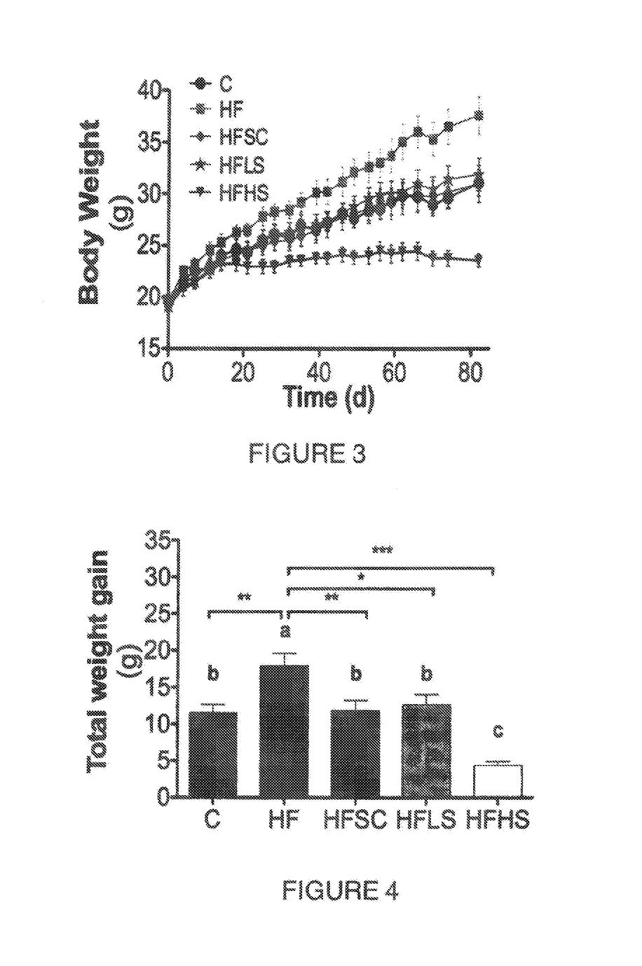

After 20 days of diet the mice weight gain started to be influenced by the different diets (FIG. 3). After 12 weeks of diet, the study was terminated. Mice fed the HF diet gained 55% more weight compared to the control group (FIG. 4). In contrast, mice fed HFSC as well as the saponin extract at the low dose (HFLS) gained weight similarly to the control counterparts despite the higher fat content. Moreover, mice fed HFHS gained less weight compared to the control and 75% less weight compared to the HF group. These results prove the effect of the saponin consumption on preventing high fat diet induced obesity.

### Example 4

A Saponin Extract that Decreases Obesity and Hyperglycemia Despite the High Fat Diet Consumption

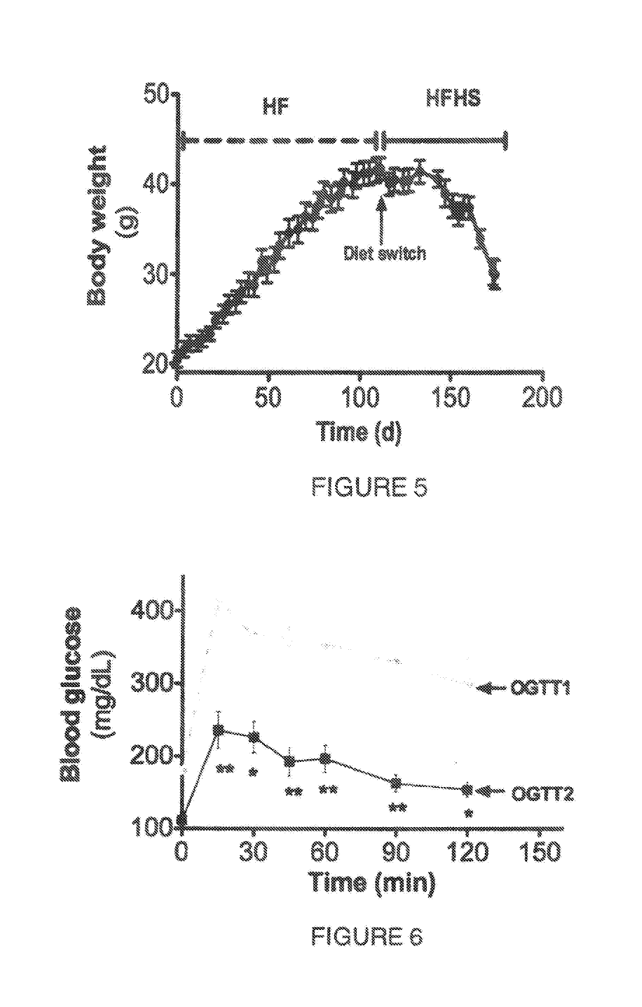

In order to assess if the consumption of the saponin extract reduced body weight and glucose intolerance in obese mice, mice (n=7) were fed with a HF diet for 16 weeks to establish obesity (FIG. 5) and hyperglycemia (FIG. 6). Subsequently, the mice were switched to the HFHS diet for another 8 weeks. An oral glucose tolerance test (OGTT) was performed at week 16 before switching the diet to HFHS (OGTT1) and a second OGTT was performed to the same mice after 8 weeks on the HFHS diet (OGTT2). To perform the OGTT, the glucose load (2 g/kg) was gavaged after 6 h of fasting. Blood glucose was determined using a blood glucose monitoring system (Abbot Laboratories, AbbotPark, Ill.), with blood samples collected from the tail vein at 0, 15, 30, 45, 60, 90, and 120 min after the glucose administration. The area under the curve (AUC) was calculated using the trapezoid rule.

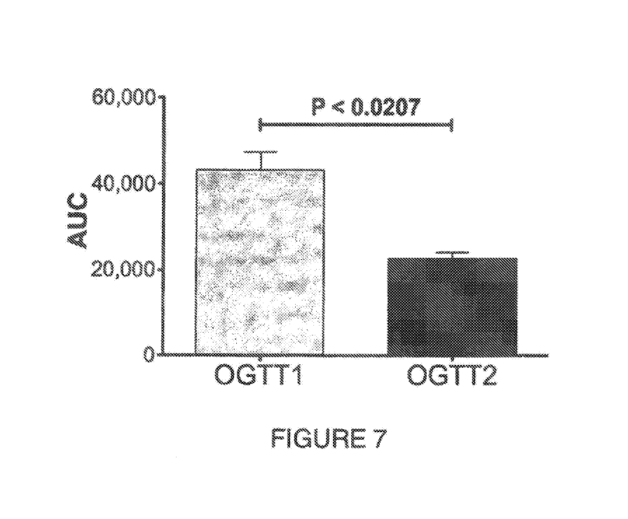

Results showed that after 16 weeks on HF diet, mice became obese (FIG. 5) and hyperglycemic (FIG. 7). After 2 weeks of HFHS diet, mice weight started to decline and after 8 weeks mice lost 28% of their body weight. In addition, the glucose tolerance was remarkably increased observed at every measurement from min 15 to min 120 of the OGTT and as a result the AUC decreased by 48% (FIG. 7) when comparing the OGTT1 vs. the OGTT2. These results prove the capability of the saponin extract to decrease obesity and improve glucose tolerance.

### Example 5

A Saponin Extract that Prevents Visceral Fat and Adipocyte Hypertrophy Despite the High Fat Diet Consumption

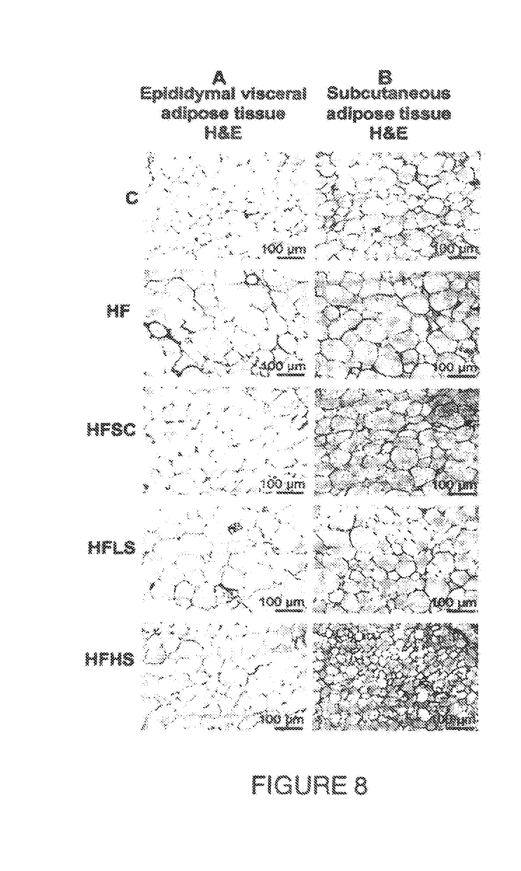

To evaluate the effect of HFLS and HFHS on the central obesity of mice fed as described in example 3, visceral fat pads (retroperitoneal and epididymal) and liver were dissected, weighted and the weight was normalized to the total mice weight. Consistent with the weight gain observed previously in mice after 12 weeks on the different diet (FIG. 4), the visceral adipose tissue weight (retroperitoneal and epididymal) was also greater in the animals fed the HF diet compared with the ones fed the control diet. Compared to the HF group, consumption of the HFHS diet prevented the increase of the epididymal and retroperitoneal visceral adiposity by 48.5% and 54.6% respectively. These results were also coherent with the adipocyte size observed in the histological cuts from the epididymal and subcutaneous adipose tissue stained with hematoxylin and eosin to visualize their morphology (FIG. 8). As observed, the consumption of HFHS diet prevented adipocyte hypertrophy compared to mice fed the HF diet. Finally, the liver weight was not affected by the experimental diets. These results prove the saponin extract capability to prevent central obesity enlargement.

### Example 6

A Saponin Extract that Prevents Hepatic Steatosis Despite the High Fat Diet Consumption

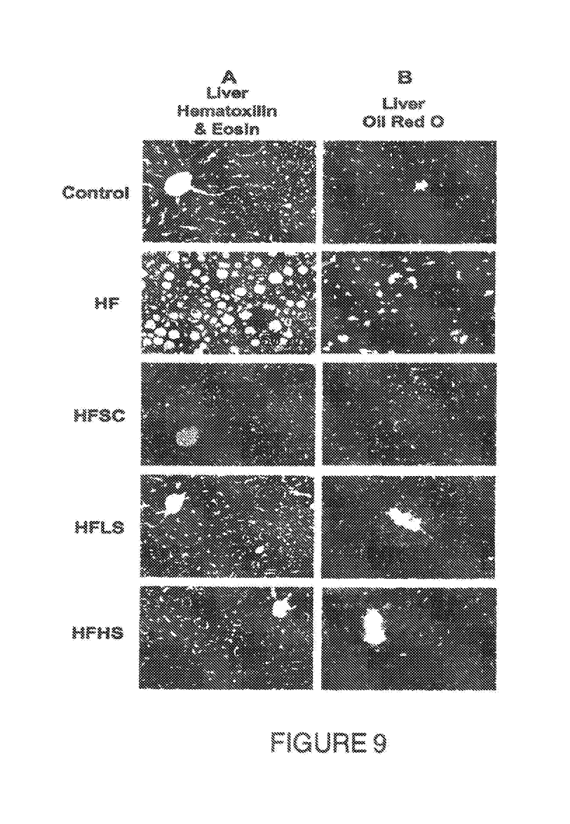

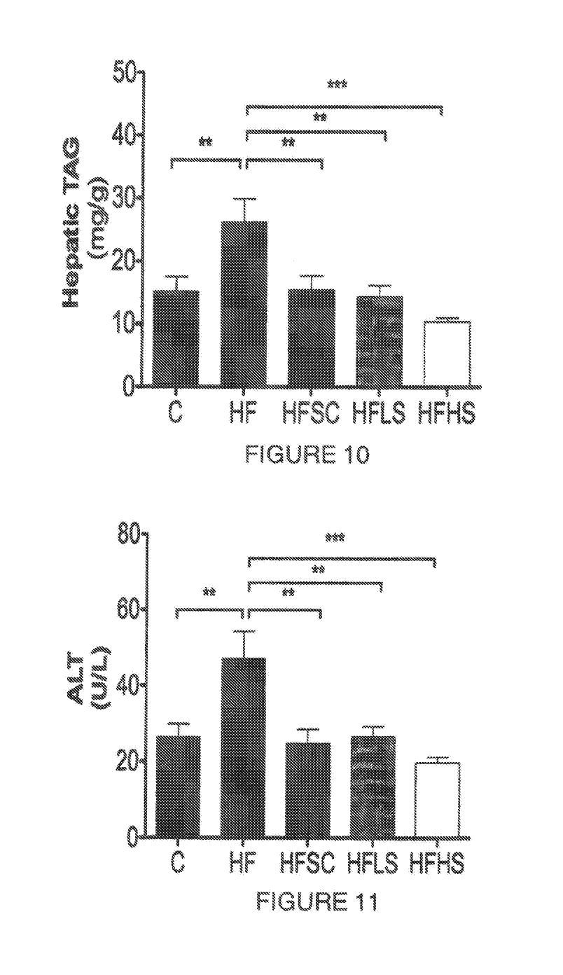

To evaluate if the effects of the saponin extract observed in example 3 also had an impact on the hepatic steatosis, the liver histopathology were evaluated using hematoxylin & eosin staining to visualize the hepatocyte morphology (FIG. 9A). To visualize the hepatic neutral lipids, cryostat frozen liver sections were stained with Oil Red O (ORO) at 0.5% in propylene glycol (Sigma-Aldrich, St. Louis, Mo.) and hematoxylin (FIG. 98). Results clearly shows that mice fed the HF diet had greater hepatic fat accumulation in the form of macro and micro vesicles compared to the control group. When agave sap concentrate was added to the HF diet (HFSC), hepatic lipid accumulation was decreased. This effect was similar with the saponins extract at the low dose (HFLS) and it was more pronounced in the saponin high dose (HFHS). The hepatic steatosis observed in the histological analysis was further confirmed by quantifying the hepatic triacylglycerides (TAG). Total hepatic lipids were extracted from homogenized liver with chloroform-methanol (2:1) and the lower phase was dried under nitrogen. The hepatic lipids were dissolved in isopropanol-Triton X-100 (10%) and assayed for and triacylglycerides concentration using an enzymatic kit (DiaSys Diagnostic Systems GmbH, Holzheim, Germany). Liver of mice fed the HF diet showed significantly (P<0.005) higher TAG accumulation compared to counterparts fed the control diet (FIG. 10). Mice fed HFSC showed a decrease in hepatic TAG content of 40.9%, and this effect was more pronounced when mice consumed the saponin extract. Hepatic TAG accumulation showed a 44.6% reduction in mice fed HFLS and 60.2% TAG reduction in mice fed HFHS compare with mice fed the HF diet.

### Example 7

A Saponin Extract with Hepato-Protective Qualities Despite the High Fat Diet Consumption

To discard hepatic damage from the previous example 6, plasma alanine aminotransferase (ALT) concentration was analyzed using a COBAS C111 analyzer (Roche, Basilea, Switzerland). As expected, consumption of a HF diet increased ALT levels compared to the control group (FIG. 11). The addition of agave sap concentrate or the saponin extract did not increase ALT content, on the contrary mice fed HFHS diet had significantly (P<0.001) less ALT concentration compared to mice fed the HF diet.

### Example 8

A Saponin Extract that Prevents Hyperleptinemia Despite the High Fat Diet

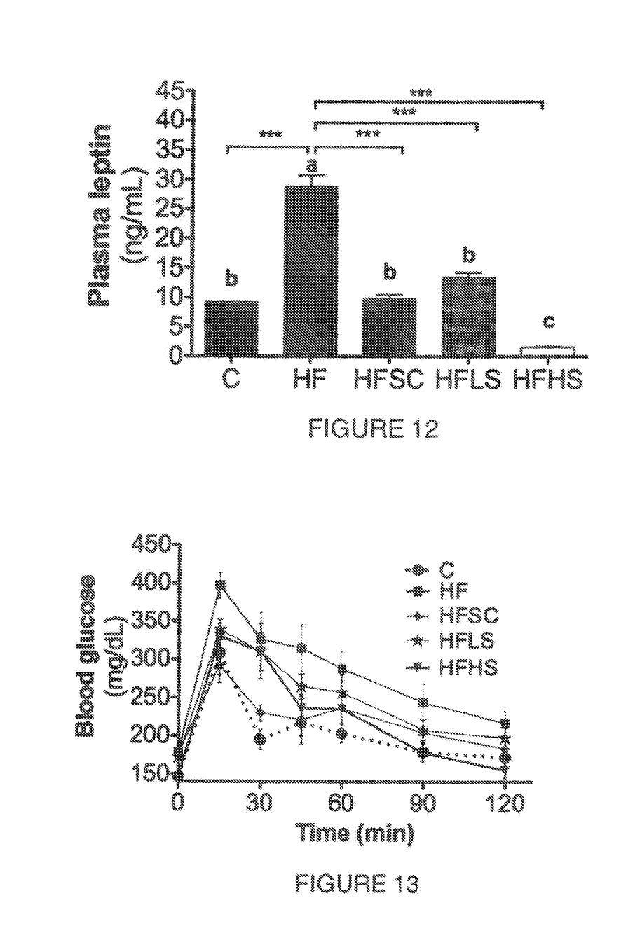

Plasma leptin concentration in animals from example 3 was quantified using a commercial ELISA kit (ALPCO, Salem, N.H.) following the manufacturers protocol. Results showed that circulating leptin increased 3-fold in mice fed HF diet compared to those fed the control diet (FIG. 12). The addition of the agave sap concentrate and the saponin extract prevented hyperleptinemia. This effect was more pronounced in mice fed HFHS compared with mice fed HFSC, where the leptin concentration decreased by 66% and 95% respectively. Circulating leptin showed a positive correlation with the visceral adipose tissue mass and adipocyte size.

### Example 9

A Saponin Extract that Prevents Hyperglycemia, Hyperinsulinema and Insulin Resistance Despite a High Fat Diet

To evaluate if the differences on the body weight gain observed in example 2 also affected the glucose clearance of the mice subjected to the different diets, an oral glucose tolerance test (OGTT) was performed at the 10^{th} week of the experiment. The OGTT was performed as described in example 4. As expected, animals fed the HF diet had a substantial increase in blood glucose compared to the control group at fasting and during the OGTT, indicating that the HF group had decreased glucose o10 tolerance (FIG. 13). Mice fed the HFHS diet significantly (P<0.05) increased the glucose tolerance calculated by a 25% smaller area under the curve (AUC) compared to mice fed the HF diet. Hyperglycemia is usually accompanied by hyperinsulinemia. Plasma samples obtained at the end of the study were analyzed for glucose and insulin concentrations. Insulin resistance was estimated indirectly through HOMA-IR, calculated as follows: (fasting glucose (mmol/L))×(fasting insulin (μU/mL))/22.5. The HF group had greater insulin concentration compared to the C group. When the saponin extract was added to the HF diet in HFHS mice group, the hyperinsulinemia was prevented. Also the HOMA-IR was lower in HFHS group compared to the HF, suggesting that saponins have an effect increasing glucose tolerance.

### Example 10

A Saponin Extract that Prevents High LDL-Cholesterol Plasma Concentration Despite a High Fat Diet.

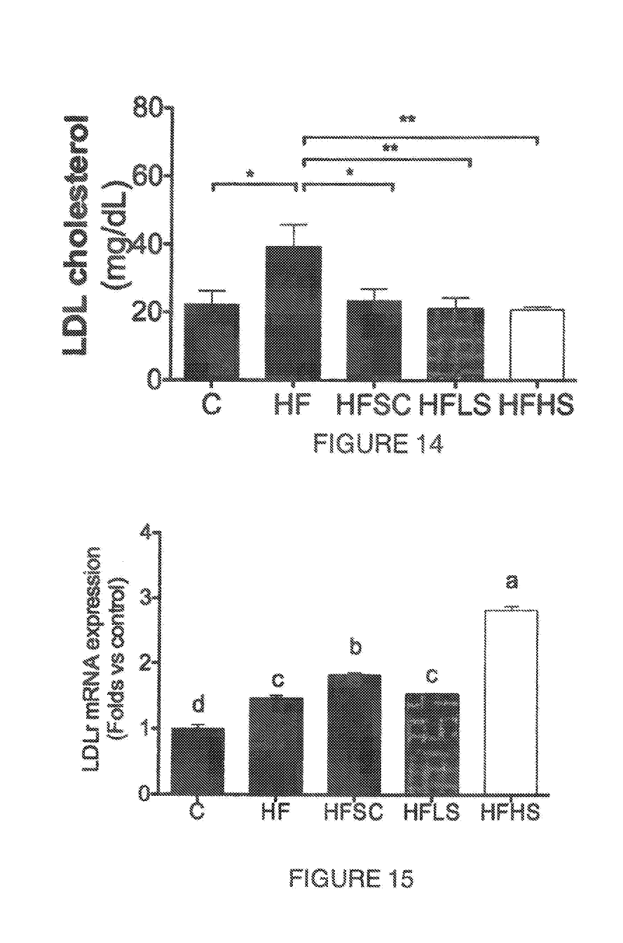

To evaluate the lipid metabolism of the mice from example 3, at the end of the study, the blood was collected and the plasma was analyzed for LDL-cholesterol using a COBAS C111 analyzer (Roche, Basilea, Switzerland). Results showed that mice fed the saponin extract had lower plasma LDL-cholesterol (LDL-C) compared to mice fed the HF diet (FIG. 14). To further analyze the hepatic gene expression, liver RNA was extracted, reverse transcription for cDNA synthesis and quantitative real-time PCR analysis were performed. Gene expression was normalized with the expression of the housekeeping gene [symbol] -2-microglobulin. Relative expression levels were calculated by the 2 [symbol] ^{Ct} metho. Gene expression results showed that the decrease in LDL-C was partially due to an overexpression of the LDL receptor (LDLr), which was overexpressed in the liver of mice fed HFHS (FIG. 15). This receptor internalizes the LDL-C cholesterol from the blood stream into the cell. Furthermore, the gen involved in the cholesterol transformation to bile acid for its excretion (cholesterol 7 alpha-hydroxylase (CYP7A1)) was up-regulated by the saponin extract, increasing 2.7-Fold in HFLS and 3.5-Fold in HFHS. Finally, the genes involved in bile acid transport for its excretion, ATP-binding cassette sub-family G member 8 (ABCG8) and ATP-binding cassette transporter ABCA1 (ABCA1) were also up-regulated by the saponin extract when compared to mice fed the HF diet and control diet.

### Example 11

A Saponin Extract that Promotes the Energy Expenditure by Increasing the Oxygen Consumption and Muscle Oxidative Fibers

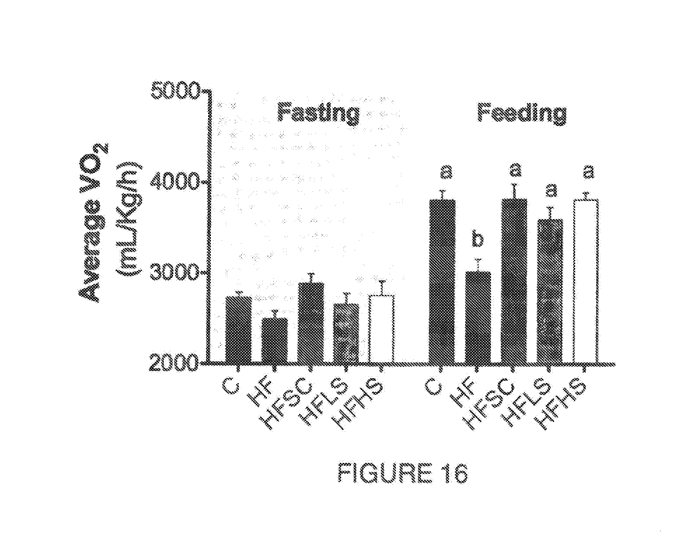

To evaluate whether changes in body weight observed in example 3 were associated with changes in energy expenditure, we performed an indirect calorimetry during the fasting and fed states. Animals were individually housed during 48 h in cages with open flow system connected to an Oxymax-CLAMS Lab Animal Monitoring System (Columbus Instruments, Columbus Ohio). Animals were acclimatized for 24 h, fasted for 6 h in the light period and fed during the dark period. Throughout the test, volume of 02 consumption (VO₂, ml/kg/h) and CO₂ production (VCO₂, mL/kg/h) were measured sequentially during 90 s. As observed in FIG. 16, the energy expenditure measured by O₂ consumption was not affected by the treatments on the fasting state, however during the fed state, mice fed the HF diet consumed 21% less O₂ compared to the control group. When agave sap concentrate (HFSC) or the saponin extract (HFLS and HFHS) were added to the HF diet, the O₂ consumption increased and was similar to the control group. Compared to the HF group, O₂ consumption from HFSC and HFHS groups increased by 27% and from HFLS group by 19%. These results demonstrated that the saponin extract increased energy expenditure despite the high-fat content of the diet.

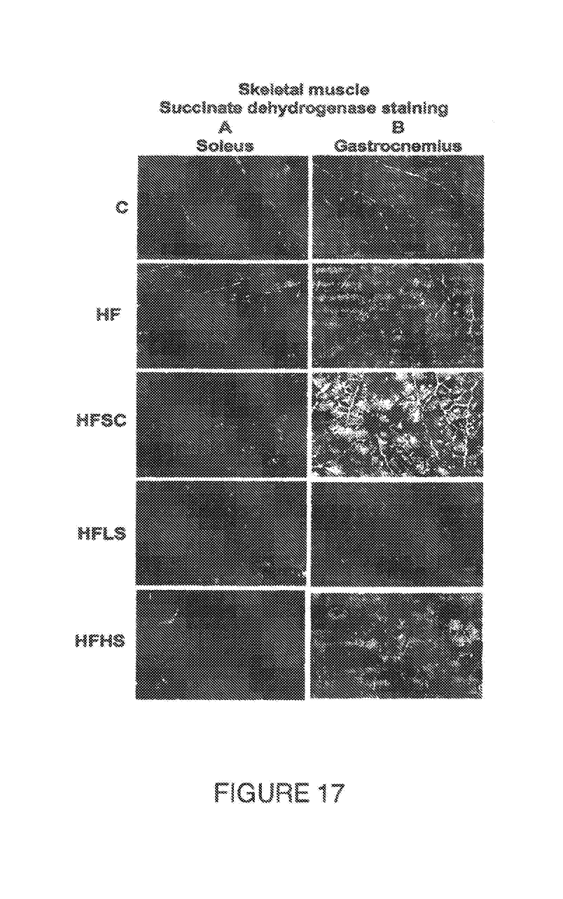

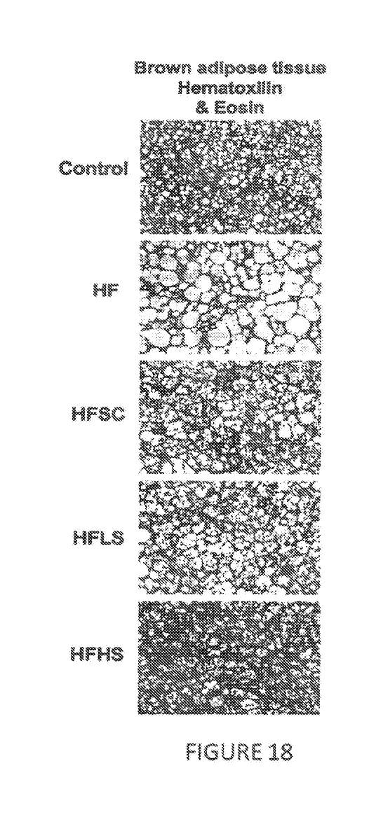

It has been suggested that energy expenditure is determined by the mitochondrial respiration mostly from the skeletal muscle. The histochemical staining for the succinate dehydrogenase enzyme is suitable to distinguish between muscle fibers with different oxidative capacities (Kalmar, Bianco, & Greensmith, 2012). Therefore, at the end of the study in week 12, gastrocnemius and soleus muscles were dissected together and immediately frozen in isopentane cooled by liquid nitrogen. Frozen sections were stained and incubated at 37° C. for 60 min. The staining solution was prepared with sodium succinate (270 mg) and nitroblue tetrazolium (10 mg) dissolved in 10 mL of 50 mM PBS (pH 7.5). Afterwards, slides were washed with deionized water and sequentially dehydrated with acetone at 30, 60 and 90%. Following, slides were rehydrated with acetone at 60, 30% and deionized water. Mounted and photographed. FIG. 17 shows that mice fed HFSC and HFLS diets had a minimal increase in SDH activity, nevertheless mice fed HFHS markedly increased SDH content. This difference was observed in the soleus as well as the gastrocnemius muscle. Greater presence of SDH is directly related with a greater content of mitochondria since this enzyme is located in the inner mitochondrial membrane. Thus, consumption of the saponin extract at the high dose increased significantly the muscle type 1 oxidative fibers and the mitochondrial abundance, consequently the muscle oxidative capacity.

Following, the oxidative genes acyl CoA oxidase (AOX) and carnitine palmitoyl transferase 1A (CPT-1A) were analyzed in the liver as described previously in the example 10. Results showed that AOX and CPT-1A were up-regulated in the mice led HFLS and HFHS diets compare the mice fed the HF diet indicating a greater utilization of the lipids by the mitochondria.

### Example 12

A Saponin Extract that Promotes Thermogenesis in the Adipose Tissue

in mammals, brown adipose tissue (BAT) can dissipate their energy as heat in a process called non-shivering thermogenesis. This tissue is essentially the primary organ for heat production (Bartelt & Heeren, 2014). Its activation confers beneficial effects on adiposity, insulin sensitivity and hyperlipidemia (Bartelt & Heeren, 2014). Different from white adipose tissue (example 5, FIG. 8), BAT is characterized by a multilocular lipid droplet structure, with high amounts of mitochondria and production of the mitochondrial uncoupling protein 1 (UCP1). To observe BAT morphology, slides were stained with hematoxylin & eosin. BAT histology (FIG. 18) clearly shows morphological alterations generated by the different diets. Mice fed the HF diet had several adipocytes with one single large lipid vacuole resembling more to WAT indicating impaired thermogenesis and lipid oxidation. On the contrary, when the saponins were added to the diet (HFLS and HFHS), the BAT morphology was similar to the control mice denoting that BAT functionality was not affected despite de high fat content in the diet.

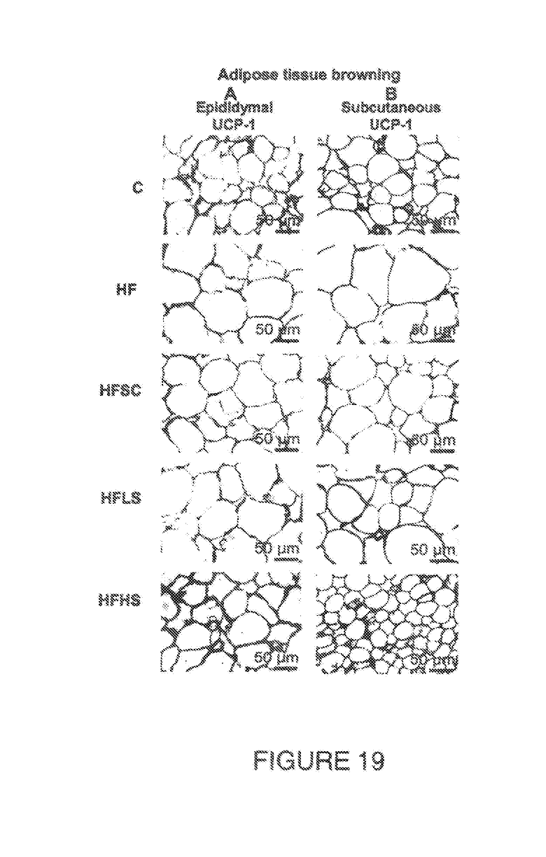

To further understand if the WAT was also affected by the saponin consumption in a process called browning, the inventors evaluated the UCP1 expression by immunohistochemistry in epididymal and subcutaneous tissue sections. Results in FIG. 19 show that UCP1 was over expressed in the adipose tissue of mice fed HFHS, observed by a darker staining indicating a browning process and an increased thermogenesis.

### Example 13

A Saponin Extract that Promotes Mitochondrial Biogenesis

To evaluate if the increase in energy expenditure of mice fed the saponin extract (example 11) was associated with greater mitochondrial activity, the inventors measured the protein expression of the peroxisome proliferator-activated receptor gamma coactivator 1- [symbol] (PGC1- [symbol] ) which is essential for transcriptional modulation of mitochondrial biogenesis and oxidative metabolism (Boström et al., 2012). To enhance PGC1-α activity, it is also necessary that 5′ adenosine monophosphate-activated protein kinase (AMPK) is activated by phosphorylation in the Thr172 residue (Jager et al., 2007).

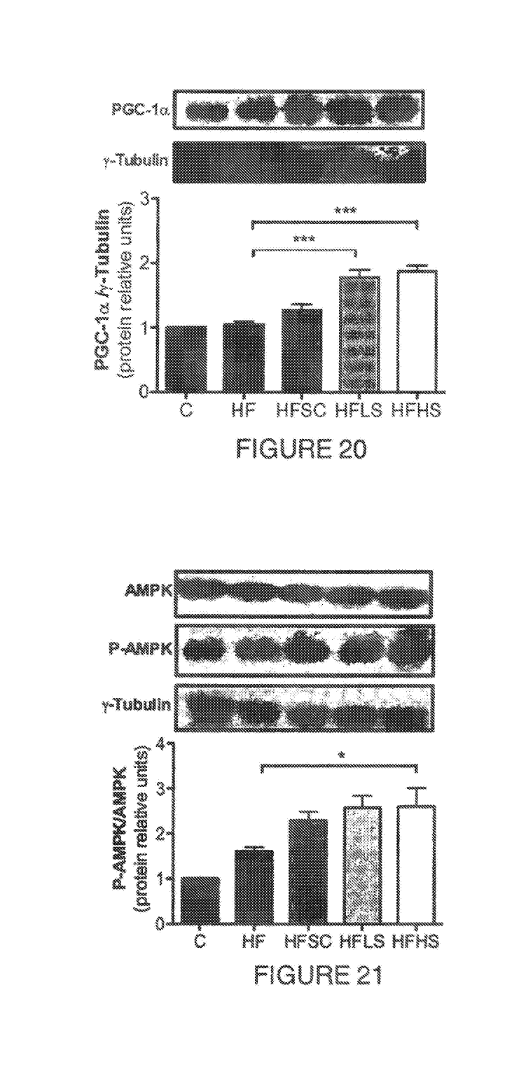

Protein expression was analyzed by western blot. To perform these analyses, protein was extracted from homogenized gastrocnemius skeletal muscle using ice-cold RIPA buffer with a Complete Mini protease inhibitor (Roche Diagnostics) and quantified with the Lowry method. Protein (20 [symbol] g) was separated in SDS-polyacrylamide gel (8%) and transferred to a PVDF membrane. The membranes were blocked for 1 h with 5% non-fat dry milk, and incubated overnight at 4° C. blocking solution with the primary antibody AMPK1/2 (1:1000), p-AMPK (Thr-172) (1:500), and PGC-10 (1:500) (Santa Cruz Biotechnologies, Santa Cruz, Calif.). The membranes were then incubated with horseradish peroxidase-conjugated secondary antibody (1:3500) for 1.5 h. Visualization was performed using a chemiluminescent detection reagent (Millipore, MA), followed by membrane exposure to film. [symbol] -Actin was used as loading control. For quantification, densitometric analyses of immunoblot bands were performed with the ImageJ software. Results showed that mice fed the control and HFSC diet had similar PGC1- [symbol] protein expression compared to mice fed the HF diet (FIG. 20). On the contrary, mice fed the saponin extract (HFLS and HFHS) showed an increment in this key regulator of the mitochondrial biogenesis and activity (Boström et al., 2012). Furthermore, the saponin extract also increased activation of the AMPK by phosphorylation in the Thr172 residue, indicating as well an increase in fatty acid oxidation (FIG. 21).

### Example 14

A Saponin Extract that Prevents Intestinal Microbiota Dysbiosis and Promotes Lactobacillus Spp., Bifidobacterium Spp. and Akkermansia muciniphila, Abundance.

To understand the effect, the saponin extract on the gut microbiota from the animals in the example 3, mice feces were collected during the 12th week of experiment. Microbial DNA was purified from the feces using a QIAamp DNA Stool Mini Kit, (Qiagen, Inc., Hilden, Germany). Microbiota analysis was performed using 16S ribosomal DNA (rDNA) to evaluated the relative abundance of the main phylas, specific genus and species related to metabolic syndrome. Microbial DNA was quantified with Real-time RT-PCR. Bacteria abundance was normalized with the expression of the 16S Universal primer and the relative abundance levels were calculated by the 2- [symbol] Ct method.

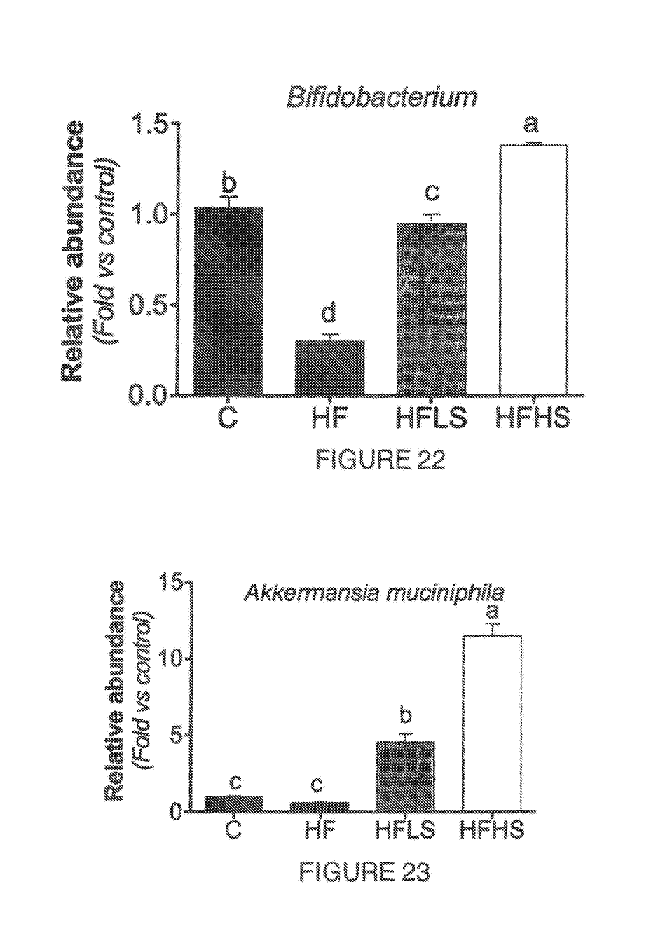

Results showed that mice fed the HF diet, the abundance of the phyla Firmicutes increased and Bacteroidetes decreased compared to the control group. In contrast, the addition of the extracted saponins prevented microbiota dysbiosis and increased the Bacteroidetes/Firmicutes ratio. The consumption of the saponin extract partially prevented the decrease of Lactobacillus relative abundance, which increased significantly compared to mice consuming the HF diet, nevertheless without reaching the control group. Additionally, mice fed HFHS significantly (P<0.05) increased the Bifidobacterium spp. relative abundance (FIG. 22). Finally, as observed in FIG. 23, Akkermansia muciniphila relative abundance was increased 4.6-fold in the HFLS group and 11.5-fold in the HFHS group. This mucin-degrading bacterium has been associated with an improvement of insulin sensitivity, decrease in fat gain and LDL cholesterol as well as increase in energy expenditure, concurring with the results previously observed.

Finally it must be understood that the Agavaceae extract comprising steroidal saponins to treat or prevent metabolic syndrome related pathologies of the present invention, is not limited exclusively to the embodiments above described and illustrated and that the persons having ordinary skill in the art can, with the teaching provided by the invention, make modifications to the Agavaceae extract comprising steroidal saponins to treat or prevent metabolic syndrome related pathologies of present invention, which will clearly be within of the true inventive concept and of the scope of the invention which is claimed in the following claims.

REFERENCES CITED PATENT DOCUMENTS

| | | | | |
|---|---|---|---|---|
| WO | WO2012/145281 | A2 | 2012 | Pan and Middleton |
| US | U.S. Pat. No. 8,357,786 | B2 | 2013 | Huh et al. |
| US | US 2014/0141108A1 | A1 | 2014 | Brown and Scott |
| RU | RU 2,559,779 | C1 | 2012 | Yakhkind et al. |
| US | U.S. Pat. No. 8,440,178 | B2 | 2013 | Darimont et al. |
| WO | WO 2014/076246 | A1 | 2014 | Cani et al. |
| US | U.S. Pat. No. 7,514,422 | B2 | 2009 | Smith and Smith |
| US | U.S. Pat. No. 6,458,924 | B2 | 2002 | Knudsen et al. |
| WO | WO2010/140165A1 | B | 2010 | Goel |
| US | US 2066/0062863 | A1 | 2006 | Ghosal |
| US | U.S. Pat. No. 7,985,846 | B2 | 2011 | Sung-Sick et al. |
| CN | CN 102,091,082 | B | 2011 | Liu et al. |
| US | U.S. Pat. No. 6,465,018 | B1 | 2002 | Tuttle |
| US | U.S. Pat. No. 7,812,004 | B2 | 2010 | Frippiat et al. |
| WO | WO 2012/066485 | A2 | 2012 | Vieyra García and |
| | | | | Pérez López |
| US | U.S. Pat. No. 8,470,858 | B2 | 2013 | Gutierrez-Uribe and |
| | | | | Serna-Saldivar |

### OTHER DOCUMENTS

Aguilar, M., Bhuket, T., Torres, S., Liu, B., & Rj. W. (2015). Prevalence of the metabolic syndrome in the United States, 2003-2012. The Journal of the American Medical Association, 313(19), 1973-1974.
American Diabetes Association. (2014). Diagnosis and classification of diabetes mellitus. Diabetes Care, 37(1). S81-S90.
Apovian, C. M., Aronne, L. J., Bessesen, D. H., McDonnell, M. E., Murad, M. H., Pagotto, U., . . . Still, C. D. (2015). Pharmacological management of obesity: An endocrine society clinical practice guideline. The Journal of Clinical Endocrinology & Metabolism, 100(2), 342-362.
Attele, A. S., Zhou, Y. P., Xie, J. T., Wu, J. A., Zhang, L., Dey, L., . . . Yuan, C. S. (2002). Antidiabetic effects of Panax ginseng berry extract and the identification of an effective component. Diabetes, 51(6), 1851-1858.
Bartelt, A., & Heeren, J. (2014). Adipose tissue browning and metabolic health. Nature Reviews. Endocrinology, 10, 24-36.
Boström, P., Wu, J., Jedrychowski, M. P., Korde, A., Ye, L., Lo, J. C., . . . Spiegelman, B. M. (2012). A PGC1- [symbol] -dependent myokine that drives brown-fat-like development of white fat and thermogenesis. Nature, 481, 463-468.
Cani, P. D., Neyrinck, a. M., Fava, F., Knauf, C., Burcelin, R. G., Tuohy, K. M., . . . Delzenne, N. M. (2007). Selective increases of bifidobacteria in gut microflora improve high-fat-diet-induced diabetes in mice through a mechanism associated with endotoxaemria. Diabetologia, 50(11), 2374-2383.
Chen. L., Tai, W. C. S., & Hsiao, W. L. W. (2015). Dietary saponins from four popular herbal tea exert prebiotic-like effects on gut microbiota in C57BL/6 mice. Journal of Functional Foods, 17, 892-902.
Dao, M. C., Everard, A., Aron-Wisnewsky, J., Sokolovska, N., Prifti, E., Verger. E. O., . . . Clément, K. (2015). Akkermansia muciniphila and improved metabolic health during a dietary intervention in obesity: relationship with gut microbiome richness and ecology. Gut Microbiota. 0, 1-11.
Everard, A., Beizer, C., Geurts, L., Ouwerkerk, J. P., Druart, C., Bindels, L. B., . . . Cani D., P. (2013). Cross-talk between Akkermansia muciniphila and intestinal epithelium controls diet-induced obesity. Proceedings of the National Academy of Sciences of the United States of America, 110, 90668-071,
Fåk, F., & Bäckhed, F. (2012). Lactobacillus reuteri prevents diet-induced obesity, but not atherosclerosis, in a strain dependent fashion in Apoe−/− mice. PLoS ONE, 7(10), 1-8.
Geiss, L S., Wang, J., Cheng, Y. J., Thompson, T. J., Barker, L., Li, Y., . . . Gregg, E. W. (2014). Prevalence and incidence trends for diagnosed diabetes among adults aged 20 to 79 years, United States, 1980-2012. Journal of the American Medical Association, 312(12), 1218.
Grundy, S. M. (2015). Adipose tissue and metabolic syndrome: too much, too little or neither. European Journal of Clinical Investigation, 1365-2362.
Harms, M., & Seale, P. (2013). Brown and beige fat: development, function and therapeutic potential. Nature Medicine, 19(10), 1252-1263.
Jager, S., Handschin, C., St. Pierre, J., & Spiegelman, B. M. (2007). AMP-activated protein kinase (AMPK) action in skeletal muscle via direct phosphorylation of PGC- [symbol] . Proceedings of the National Academy of Sciences of the United States of America, 104(29), 12017-12022.
Kalmar, B., Blanco, G., & Greenesmith, L. (2012). Determination of muscle fiber type in rodents in: Current Protocols in Mouse Biology. (Vol. 2). Hoboken, N.J., USA: John Wiley & Sons. Inc.
Lagouge, M., Argmann, C., Gerhart-Hines, Z., Meziane. H., Lerin, C., Daussin, F., . . . Auwerx, J. (2006). Resveratrol improves mitochondrial function and protects against metabolic disease by activating SIRT1 and PGC- [symbol] . Cell, 127(6), 1109-1122.
Leal-Díaz, A. M., Santos-Zea, L., Martínez-Escobedo, H. C., Guajardo-Flores, D., Gutiérrez-Uribe, J. A., & Sema-Saldivar, S. O. (2015). Effect of Agave americana and Agave salmiana ripeness on saponin content from aguamiel (agave sap). Journal of Agricultural and Food Chemistry, 63, 3924-3930.
Lee, C. E., Hur, H. J., Hwang, J.-T., Sung, M. J., Yang, H. J., Kim, H.-J., . . . Kim, M.-S. (2012). Long-Term consumption of Platycodi radix ameliorates obesity and insulin resistance via the activation of AMPK pathways. Evidence-Based Complementary and Alternative Medicine, 2012, ID 759143.
Ley, R., Turnbaugh, P., Klein, S., & Gordon, J. (2006). Microbial ecology: human gut microbes associated with obesity. Nature, 444(7122), 1022-3.
Liu, M., Xu. L., Yin, L., Qi, V., Xu, Y., Han, X., . . . Peng, J. (2015). Potent effects of dioscin against obesity in mice. Scientific Reports, 5, 7973.
Macías, F. A., Guerra, J. O., Simonet, A. M., & Nogueiras, C. M. (2007). Characterization of the fraction components using 1D TOCSY and 1D ROESY experiments. Four new spirostane saponins from Agave brittoniana Trel. spp. Brachypus. Magnetic Resonance in Chemistry, 44, 615-620.
Mitra, A., & Bhattacharya. D. (2006). Dose-dependent effects of Fenugreek composite in Diabetes with dislipidaemia. Internet Journal of Food Safety, 8, 49-55.
Omoruyi, F. O. (2008). Jamaican bitter yam sapogenin: potential mechanisms of action in diabetes. Plant Foods for Human Nutrition, 63(3), 135-40.
Pan, H., Guo, J., & Su, Z. (2014). Advances in understanding the interrelations between leptin resistance and obesity. Physiology & Behavior, 130C, 157-169.
Reeves, P. G., Nielsen. F. H., & Fahey, G. C. J. (1993). AIN-93 purified diets for laboratory rodents; final report of the American Institute of Nutrition ad hoc writing committee on the reformulation of the AIN-TA rodent diet. The Journal of Nutrition, 123(11), 1939-1951.
Rendön-Huerta, J. A., Juárez-Flores, B., & Delgado-Portales, R. E. (2012). Effects of different sources of fructans on body weight, blood metabolites and fecal bacteria in normal and obese non-diabetic and diabetic rats. Plan Foods and Human Nutrition, 67, 64-70.
Roberfroid, M. B. (2000). Prebiotics and probiotics: are they functional foods? The American Journal of Clinical Nutrition, 71, 1682-1687.
Santos, A. P., Rogero, M. M., & Bastos, D. H. M. (2010). Edible plants, their secondary metabolites and antiobesogenic potential. Recent Patents on Food, Nutrition & Agriculture, 2(3), 195-212.
Santos-Zea, L., Leal-Díaz, A. M., Cortés-Cebalos, E., & Gutiérrez-Uribe, J. A. (2012). Agave (Agave spp.) and its traditional products as a source of bioactive compounds. Current Bioactive Compounds, 8(3), 218-231.
Schindhelm, R. K., Diamant, M., Dekker. J. M., Tushuizen. M. E., Teerlink, T., & Heine, R. J. (2009). Alanine aminotransferase as a marker of non-alcoholic fatty liver disease in relation to type 2 diabetes mellitus and cardiovascular disease. Diabetes/Metabolism Research and Reviews, 22. 3-12. http://doi.org/10.1002/dmrr
Tremaroii, V., & Backhed, F. (2012). Functional Interactions between the gut microbiota and the host metabolism. Nature, 489, 242-249. http://doi.org/10.1038/nature11552
USDA. (2015). Agave L search in: Plants Database. Retrieved from http://plants.usda.gov/ on October 2015.
Xiong. Y., Shen, L., Liu, K. J., Tso, P., Xiong, Y., Wang, G., . . . Lu, M. (2010). Antiobesity and antihyperglycemic effects of ginsenoside Rb1 in rats. Diabetes, 59(10), 2505-2512.
Yun, J. W. (2010). Possible anti-obesity therapeutics from nature—a review. Phytochemistry, 71(14-15), 1825-18641.

1-67. (canceled)

68. An extract from plants of the Agavaceae family comprising steroidal saponins, wherein said extract has beneficial effects on the organism of mammals in relation to the prevention or treatment of metabolic disorder related pathologies in mammals, including humans and further beneficial effects on lipid metabolism, glucose metabolism, energy expenditure and gut microbiota health.

69. An extract as claimed in claim 68, wherein the concentration of steroidal saponins in the extract is of from 30 to 90% in weight.

70. An extract as claimed in claim 68, wherein the most abundant saponins present in the extract are kammogenin glycosides, comprising >30% of the total saponin and sapogenin concentration and the sapogenin concentration is at least 0.01%.

71. An extract as claimed in claim 68, further comprising at least one saponin from the group comprising: agamenoside, agaveside, agavoside, magueyside, agavasaponin, cantalasaponin, sisalsaponin, gabrittonoside, dongnoside, amolonin.

72. An extract as claimed in claim 68, further comprising at least one aglycone from the group comprising: kammogenin, manogenin, gentrogenin, hecogenin, tigogenin, chlorogenin, sarsapogenin, gitogenin.

73. An extract as claimed in claim 68, further comprising phytochemicals such as alkaloids, polyphenols, flavonoids, phytosterols, triterpenes, policosanols.

74. A composition comprising the extract of claim 68, wherein said composition has beneficial effects on the organism of mammals in relation to the prevention or treatment of metabolic disorder including metabolic syndrome, diabetes and their related pathologies in mammals and further beneficial effects on lipid metabolism, glucose metabolism, energy expenditure and gut microbiota health.

75. A composition as claimed in claim 74, wherein the amount of extract contained in the composition is of from 0.001 to 70% in weight.

76. A composition as claimed in claim 74, wherein the amount of saponins contained in the composition is preferably of from 0.001 to 70% in weight.

77. A method for the treatment or prevention of metabolic disorder including syndrome, diabetes and their related pathologies in mammals, including humans, said method comprising administering to the mammal in need of such treatment and/or prophylaxis, an effective and/or prophylactic amount of the extract of claim 68.

78. A method to benefit the lipid metabolism in mammals, including humans said method comprising administering to the mammal in need of such treatment and/or prophylaxis, an effective and/or prophylactic amount of the extract of claim 68.

79. A method to benefit the glucose metabolism in mammals, including humans, said method comprising administering to the mammal in need of such treatment and/or prophylaxis, an effective and/or prophylactic amount of the extract of claim 68.

80. A method to benefit energy expenditure in mammals, including humans, said method comprising administering to the mammal in need of such treatment and/or prophylaxis, an effective and/or prophylactic amount of the extract of claim 68.

81. A method to benefit gut microbiota health in mammals, including humans, said method comprising administering to the mammal in need of such treatment and/or prophylaxis, an effective and/or prophylactic amount of the extract of claim 68.
